(19) United States
(12) Patent Application Publication
(10) Pub. No.: US 2006/0105345 A1
(43) Pub. Date: May 18, 2006

(54) Method for diagnosing lung transplantation rejection

(75) Inventor: Seyedhossein Aharinejad, Vienna (AT)

Correspondence Address: HELLER EHRMAN WHITE & MCAULIFFE LLP, 1717 RHODE ISLAND AVE, NW, WASHINGTON, DC, 20036-3001, US

(73) Assignee: Tim Italia S.p.A

(21) Appl. No.: 10/548,828

(22) PCT Filed: Mar. 4, 2004
(86) PCT No.: PCT/EP04/02150

§ 371 Date: Nov. 2, 2005

(30) Foreign Application Priority Data
Mar. 18, 2003 (AT) A 429/2003

(51) Int. Cl. C12Q 1/68; G01N 33/537

(52) U.S. Cl. 435/6; 435/7.92

(57) ABSTRACT

The invention relates to a method for diagnosing lung transplantation rejection comprising determining the amount of Hepatocyte Growth Factor (HGF) in a body fluid or tissue sample of a patient who has undergone lung transplantation.

The present invention relates to a method for diagnosing lung transplantation rejection.

Since its inception (1) , tremendous progress has been achieved in lung transplantation (2-4). Currently, chronic obstructive pulmonary diseases (COPD), including emphysema and α-1 antitrypsin deficiency syndrome, followed by cystic fibrosis, idiopathic pulmonary fibrosis, and primary pulmonary hypertension, are the most common indications for lung transplantation (3-5). The most important causes of morbidity and mortality in lung transplantation are primary graft failure, infection, and acute and chronic rejection (4). Surveillance protocols involving transbronchial lung biopsies (TBB) have shown that most transplant recipients have at least one episode of acute rejection, the incidence of which is greatest within the first 100 days after transplantation (4, 6, 7).

Clinical criteria alone are imprecise to diagnose rejection. TBB can diagnose graft rejection, but is invasive and impractical to perform repeatedly (7). Missing early rejection increases morbidity and mortality, and has prompted a search for markers of rejection. An ideal marker should be a) noninvasively obtained, b) repetitively useable, c) sensitive, d) easily and quickly performed, and e) inexpensive. A serologic marker would best fit these criteria and could compliment clinical parameters (7) to more accurately and quickly recognise graft rejection. Serum adhesion molecules and broncho-alveolar lavage (BAL) markers have proved to be disappointing markers (8, 9, 10) and none has become a standard test.

Evidently, the need for such a marker for easy and reliable diagnosis of lung transplant rejection exists. It is therefore an object of the present invention to provide such a suitable marker. The test system should also enable an early detection of graft rejection.

Therefore the present invention provides a method for diagnosing lung transplantation rejection comprising determining the amount of Hepatocyte Growth Factor (HGF) in a body fluid or tissue sample of patients who have undergone lung transplantation. It has been shown with the present invention that a significantly elevated level of HGF in such patients is indicative of lung transplant rejection. The present invention provides a fast and reliable tool for diagnosing lung graft rejection even in a very early post-operative phase (e.g. as early as day 2 or 3) , which allows a quick administration of adequate therapy (e.g. steroid therapy) to prevent graft failure that may result in retransplantation or patient death. It is especially the possibility of an early onset of e.g. steroid therapy, which represents a major advantage of the present invention, since the success rate of such a steroid therapy is essentially dependent on the (early) timing of the therapy. Moreover, with the present invention a diagnosis using further invasive or surgical steps (such as TBB) can be avoided. In addition, the present invention can serve as an easy monitoring in lung transplantation patients, considering that routine serum tests are performed repeatedly in these patients during their post-operative hospitalisation period, as well as at surveillance time points (to measure parameters like blood gases, kidney and liver enzymes, etc.). Therefore, the HGF levels can be easily detected in these serum samples.

The method according to the present invention generally comprises in a standard procedure the steps of
providing a body fluid or tissue sample which has been obtained from a patient after lung transplantation,
determining the amount of HGF in these samples,
comparing the determined amount of post-transplant HGF to a standard amount of HGF and
diagnosing a lung transplant rejection, if the determined amount of post-transplant HGF is significantly elevated and therefore indicative of such an event.

HGF was first detected in the plasma of partially hepatectomised rats (11) ; and later purified from human plasma and lung fibroblasts (12, 13). In addition to its role in regeneration of an injured lung (14) , HGF acts in other organs and recent studies in animals show that it might be a sensitive measure of kidney rejection (15, 16). However, clinical data provided in the course of the present invention (see “Comparative Example” in the example section) show that HGF levels can not be used to predict and analyse kidney transplant rejection in humans. The role of HGF as a marker in humans for kidney or for other organs, such as lung, was therefore completely unpredictable. Surprisingly, HGF proved to be a suitable and reliable marker for (early and late) lung transplantation rejection.

The body fluid or tissue sample in which the HGF amount is determined according to the present invention may be from any source in which the elevated HGF level is indicative of lung transplantation rejection, e.g. sources being selected from the group comprising serum, plasma, blood, transsudates, lung or bronchial drainages or lavages, sputum, urine, lung adjacent tissue and lung tissue. Since obtaining tissue samples may be associated with the disadvantages already known for the methods currently being applied (TBB), non-invasive sampling methods from the patient bypassing invasive (operative) burden for the lung are preferred: serum, plasma, blood, transsudates in the intra-operatively implanted drains, lung or bronchial lavages and sputum.

Preferably the method according to the present invention comprises the following steps:
providing a body fluid or tissue sample of a patient who has undergone lung transplantation,
determining the amount of HGF in said body fluid or tissue sample,
comparing whether said determined amount of HGF is elevated in comparison with the amount of HGF in patients without lung transplantation rejection and
a) diagnosing lung transplantation rejection, if said determined amount of HGF is elevated in comparison with the amount of HGF in patients without lung transplantation rejection or
b) not-diagnosing lung transplantation rejection, if said determined amount of HGF is not elevated in comparison with the amount of HGF in patients without lung transplantation rejection.

The way in which the amount of HGF is determined in the sample is not critical, numerous methods are well known in the art; for medical reasons such a determination has to be standardised. Preferred methods or means are selected from the group ELISA, RIA, mass spectroscopy, affinity chromatography, flow cytometry, protein-microarrays, fluorescent antibodies, HGF binding substances, especially monoclonal and polyclonal HGF-antibodies or HGF receptors, immunohistochemical methods, immunoprecipitation, Western blot, fluorescence resonance energy transfer (FRET), cDNA array and nucleic acid amplification methods (the latter of course only for tissue samples wherein elevated HGF mRNA may be indicative for lung transplantation events). Examples for HGF binding substances and methods for determining HGF levels in samples of patients are described in (27-38). However, it is clear to the skilled man in the art that the ways and means by which HGF levels are determined is not critical for the present invention. Only the determination of the amount of HGF by a binding event with the HGF binding substance should be enabled with the necessary specificity (i.e. being able to discriminate between amounts of the groups “rejectie” and “non-rejectie”).

The elevated HGF amount being indicative of lung transplantation rejection according to the present invention may preferably be ascertained by at least one of the following:
the determined HGF amount is at least 400%, preferably at least 500%, especially at least 600%, of the mean baseline level of HGF in healthy controls,
the determined HGF amount is at least 200%, preferably at least 300%, especially at least 350%, of the mean baseline level of HGF in patients before lung transplantation,
the determined HGF amount is at least 200%, preferably at least 300%, especially at least 350%, of the mean baseline level of HGF in patients on day 3 or later after lung transplantation with no events, especially with no transplantation rejection and/or with no infection.

Depending on the way of measuring the amount of HGF, the patient or group sample or the source of sample, such comparative levels of HGF indicating “no transplant rejection”, the absolute and relative values may differ, however, HGF has proven to be an excellent and significant marker so that the skilled specialist in the art can easily apply the teachings of the present specification to determine whether a HGF level is elevated in the transplantation patient or not.

For example, the above mentioned mean baseline levels may be given in the following ranges (in serum):

mean baseline level of HGF in healthy controls: between 300 and 900 pg/ml serum;

mean baseline level of HGF in patients before lung transplantation: between 900 and 1600 pg/ml serum;

mean baseline level of HGF in patients on day 3 after lung transplantation with no events, especially with no transplantation rejection and/or with no infection: between 800 and 1500 pg/ml serum.

In absolute values (also being highly dependent on the HGF determination method) an elevated HGF amount being indicative of lung transplantation rejection may preferably be ascertained by a serum level preferably at day 3 (or later) after lung transplantation of higher than 2000 pg/ml, preferably higher than 2300 pg/ml, especially higher than 2500 pg/ml. Serum HGF levels in patients with lung transplant rejection can raise between 3000 pg/ml to 5000 pg/ml or more.

A preferred method to measure HGF levels is to monitor the levels in the same patient before the transplantation and thereafter. Thereby a (slightly or even pronounced) elevated level of HGF will be observed shortly after (a few hours or about one or two days) surgery that will, however, go down again to pre-surgical levels or even below and stay there, if rejection events do not occur. A significant post-surgical rise in HGF levels or a lack of post-surgical decrease is indicative of rejection events; a slight rise on day 3 or later may be indicative of an infection.

According to another aspect, the present invention provides the use of HGF binding substances for measuring the amount of HGF in a body fluid or tissue sample provided from a patient who has undergone lung transplantation. HGF binding substances may be any substance that shows a specific affinity for HGF by which a determination of HGF is enabled, such as HGF antibodies (including of course all fragments and derivatives thereof exhibiting the same specificity) or HGF receptors.

According to another aspect, the present invention further relates to a kit for diagnosing lung transplantation rejection comprising
a sample container with a body fluid or tissue sample of a patient who has undergone lung transplantation and
means for determining the amount of HGF in said sample,
or the use of a kit for determining the amount of HGF for diagnosing lung transplant rejection.

The kit according to the present invention further comprises comparative HGF preparations containing a determined amount of HGF, e.g. plasma samples, HGF concentration series, HGF calibration curves, etc.

Preferably the kit according to the present invention is provided in the form of a (micro) plate, biochip, membrane, i.e. where the means for determining the amount of HGF comprises a (micro) plate, biochip, or membrane, e.g. with HGF capturing molecules and/or HGF (standards) being immobilised on a solid surface.

The present invention is further illustrated by the following examples and the drawing figures, yet without being restricted thereto.

### DESCRIPTION OF THE FIGURES

FIG. 1: Mean HGF serum levels in controls, and in COPD, cystic fibrosis (CF), and idiopathic lung fibrosis (LF) patients before (Pre-OP) transplantation. HGF levels are elevated but not significantly different in all patient groups as compared with controls.

FIG. 2: Mean HGF serum levels in controls, pre-transplant patients (Pre-OP), and in patients with no events up to 24 weeks after transplantation. The mean serum HGF levels in patients with no events significantly increased on the first and second post-operative day vs. controls, decreased and remained stable at levels, which were not significantly different vs. controls. (d) days and weeks (w) after transplantation. *, significantly different vs. control levels (p<0.01) ; †, significantly different vs. pre-transplant levels (p<0.05).

FIG. 3: Mean HGF serum levels in controls, in patients before transplantation (Pre-OP), in patients after transplantation with no events (No event), in patients with rejection before treatment (Rejection), in patients with rejection at the end of steroid treatment (rejection CTRL), in patients with infection before treatment (Infection), and in patients with infection at the end of treatment (infection CTRL). HGF levels significantly increased at rejection vs. all groups († p<0.01 vs. all groups). * p<0.05 vs. controls.

FIG. 4: Graphic shows HGF serum levels in a patient 4 days before and after graft rejection (▪) was diagnosed clinically (⇓, day 0). Serum levels in one patient with no events (∘) and in one patient with bacterial infection (Δ) are plotted to demonstrate the sensitivity of HGF serum levels to predict graft rejection. Serum HGF levels increase significantly to 5000 pg/ml only in the patient with graft rejection one day before the diagnosis was made clinically and verified by TBB.

FIG. 5: Mean HGF serum levels in controls, in patients after renal transplant surgery with no events, and in patients with diagnosed renal graft rejection. HGF does not change in rejection vs. patients with no events after transplantation (* p<0.05 vs. controls).

### EXAMPLES

Patients and Methods:

Patients: Eighty six patients undergoing lung transplantation who gave informed consent to participate were included in this study. Twelve healthy, age and sex matched volunteers served as controls. Table 1 summarises the demographic data of the individuals included in the study. Serum samples were collected and coded before and after lung transplantation daily for the first week, then every two days from second to the fifth week, followed by weekly up to 24 weeks after transplantation. Episodes of rejection and infection, including the treatment protocols and time table of these events were determined (Table 2) , and were analysed and correlated to coded HGF serum levels at the end of the study.

TABLE 1
Number, age, gender, and the diagnosis of patients and controls

| | | Age | | |
|---|---|---|---|---|
| Diagnosis | Patients (n) | Mean ± SD | Range | Sex |
| COPD | 53 | 54.3 ± 8.9 a | 16-68 a | 30M/23F |
| Cystic fibrosis | 17 | 25.3 ± 11.1 a | 12-45 a | 6M/11F |
| Idiopathic lung | 16 | 52.4 ± 10.5 a | 36-67 a | 8M/8F |
| fibrosis | | | | |
| Controls | 12 | 29.1 ± 8.9 | 14-42 a | 7M/5F |

TABLE 2
Time course of rejection and infection episodes

| | Events | | | | | |
|---|---|---|---|---|---|---|
| Diag- | Infec- | | | Rejec- | | |
| nosis | tion | POD* | Sex | tion † | POD | SEX |
| COPD | Bacteri- | 7, 9, 10, | 11M/5F | 15 | 4, 5, 8, | 11M/4F |
| | al, | 12, 16, 19, | | | 11, 14, | |
| | fungal | 21, 27, 29, | | | 14, 16, | |
| | | 33, 33, 34, | | | 17, 20, | |
| | | 34, 38, 40, | | | 21, 21, | |
| | | 41, 44, 54, | | | 32, 46, | |
| | | 54, 56, 71, | | | 94, 131 | |
| | | 77, 85, 102 | | | | |
| Cystic | Bacteri- | 7, 7, 12, | 3M/6F | 4 | 4, 9, 16, | 1M/3F |
| fi- | al, | 16, 18, 21, | | | 22 | |
| brosis | fungal | 28, 31, 37, | | | | |
| | | 40, 51, 52, | | | | |
| | | 69, 76 | | | | |
| Idio- | Bacteri- | 8, 13, 19, | 3M/1F | 3 | 11, 18, 20 | 2M/1F |
| pathic | al, | 24, 25, 34, | | | | |
| lung | fungal | 41 | | | | |
| fi- | | | | | | |
| brosis | | | | | | |

*Post-operative day;
† Number of rejections

Diagnosis and treatment of events: Rejection and infection were diagnosed based on TBB, BAL, chest radiographs, blood gas analysis, pulmonary function tests, and leukocyte count. Surveillance TBB was performed at weeks 3, 6, 12, and 24 after transplantation or when clinically indicated. Rejection was diagnosed clinically if patients had new radiographic pulmonary shadows and deterioration of blood gas and pulmonary function without evidence of infection, and histologically if TBB proved rejection grade 2 or higher according to International Society for Heart and Lung Transplantation scoring.

Immunosuppressive treatment: During surgery, 500 mg methylprednisolone was given prior to revascularisation of the graft and was cycled every 8 h at 125 mg for one day. Prednisolone was given at 1 mg/kg/d, starting on day 2 and reduced to 0.5 mg/kg/d over the first week. This dose was tapered to 0.15-0.3 mg/kg/d over the following 6 months. Antithymocyte globulin (Thymoglobuline, Sero-Meriux, France) was administered as induction therapy at 2.5 mg/kg/d for the first 4 days after transplantation. Cyclosporine A (Sandimmun, Novartis, Basel, Switzerland) was started i.v. when the recipient was stable hemodynamically after transplantation. Cyclosporine A administration was then switched to oral and adjusted progressively to a target serum levels of 250-350 ng/ml for the first month, and to 200-250 ng/ml thereafter. Mycophenolate mofetil (Cellcept, Hofmann-La Roche, Basel, Switzerland) was administered twice at 1 g within 24 h after transplantation.

HGF assay: Enzyme linked immunosorbent assay (ELISA) for HGF (Quantikine™, R&D Systems, Minneapolis, Minn.) was performed according to manufacturer's protocol. A HGF monoclonal antibody was pre-coated onto a microplate and 100 μl of assay diluent (buffered protein base) was added to each well, followed by 100 μl of standard and the serum sample. The wells were incubated for 2 h at room temperature, then aspirated and washed. HGF antibody conjugated to horseradish peroxidase (200 μl) was added and incubated for 2 h at room temperature followed by three washes. Thereafter, 200 μl substrate solution (stabilised hydrogen peroxide; stabilised chromogen and tetramethylbenzidine) were added to each well and incubated for 25 minutes at room temperature to develop colour. The reaction was stopped by adding 50 μl of 2 N sulfuric acid. The optical density was detected at 450 nm.

Statistical Analysis: At the end of the study, the key was broken, the clinical parameters as well as HGF serum levels were plotted over time and analysed using ANOVA with Bonferoni t-test. P values<0.05 were considered as significant.

Results:

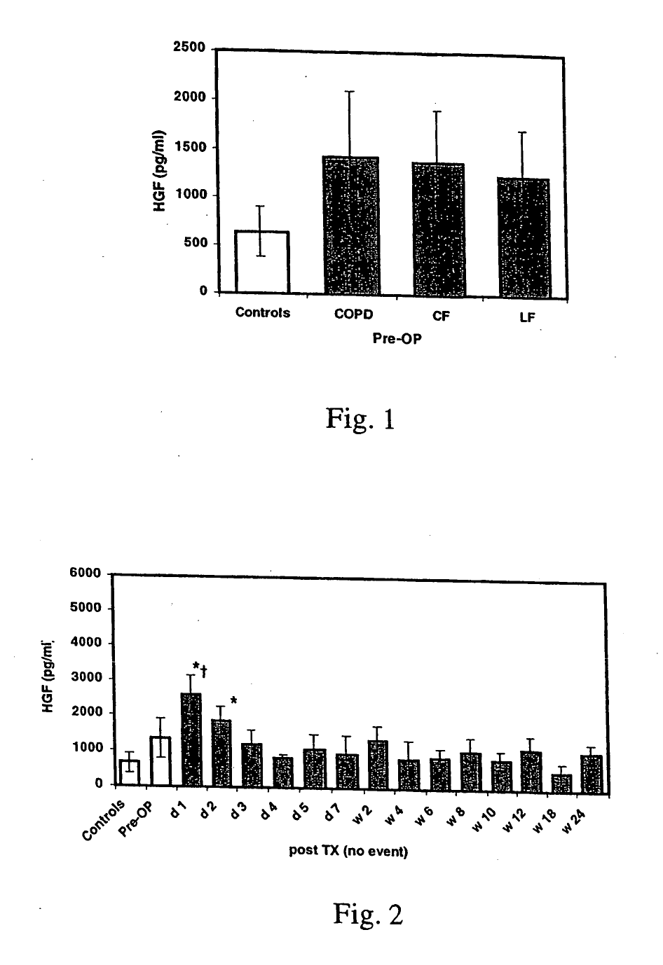

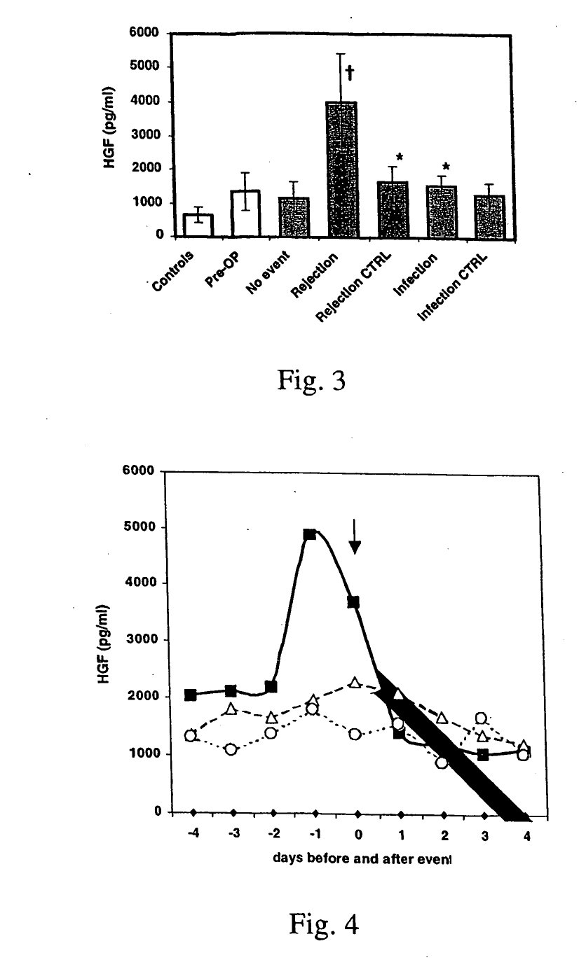

The mean serum HGF levels were 645±259 pg/ml in controls and 1425±676 pg/ml, 1376±540 pg/ml, and 1239±489 pg/ml in pre-transplant COPD, cystic fibrosis, and idiopathic lung fibrosis patients, respectively (not significant; FIG. 1). To understand how HGF serum levels would change following lung transplantation, serum HGF levels were plotted over time in all patients who had no events after surgery. The mean serum HGF level in these patients increased to 2584±548 on the first post-operative day (p<0.01 vs. controls; p<0.05 vs. pre-transplant), decreased continuously to 930±537 on day 7 (FIG. 2). Except for not significant changes when compared to day 7, HGF serum levels remained stable and reached 1034±267 in the 24th post-operative week (not significant vs. pre-transplant serum HGF levels; FIG. 2). Next, mean serum HGF levels in transplanted patients with rejection or infection both before and after treatment, were compared to those in all groups. Serum HGF levels increased significantly in patients with rejection (3978±1435) as compared with its levels in controls, in pretransplant patients and in patients with no events (p<0.01). Following steroid treatment, serum HGF levels of patients with graft rejection decreased almost to pre-operative levels and remained stable (FIG. 3). Infection increased slightly but not significantly the patients HGF serum levels (1543±303) vs. patients who had no events as well as vs. controls. HGF values in patients diagnosed for graft rejection significantly increased one day before the diagnosis rejection was made clinically and verified by TBB, while increase of serum HGF-levels associated with infections were not significant (FIG. 4).

Discussion:

Many studies have focused on the role of cytokines and growth factors in development of inflammatory responses (17) , graft dysfunction (18) , and graft rejection (19) following lung transplantation. Evidence is provided that serum levels of certain members of interleukin family are increased during early hemodynamic failure after lung transplantation (17). Transforming growth factor beta may be important in chronic lung graft rejection with bronchiolitis obliterans, because its mRNA levels are increased in patients with bronchiolitis obliterans (19). On the other hand gamma-Interferon gene expression associated with ICAM-1, is increased in BAL fluid of patients with allograft dysfunction after lung transplantation. However, serum adhesion molecules have proved to be disappointing serologic markers (8) and BAL markers may be more useful (9, 10) but none has become a standard test.

This first study on serum levels of HGF in lung-transplanted patients shows that HGF serum levels significantly increase shortly before graft rejection occurs. Serum HGF levels rise in fulminate hepatitis (20) and evidence exists that HGF levels increase in patients with inflammatory lung diseases (21). Why HGF should go higher with acute rejection than with infection is uncertain. Although both acute rejection and infection involve inflammation, rejection may involve a wider range of mediators and be more likely to involve the processes of growth and remodeling, which are closely associated with chronic rejection. Another explanation for this phenomenon might be that the stimuli that cause an increase in HGF are likely to be more focal than the many factors associated with the process of rejection and the many parameters that go into measuring it (1, 3, 6). The present invention shows that HGF is a pulmotrophic molecule and its serum levels are associated with the degree of lung injury and repair. In all cases of graft rejection in this study, the HGF serum levels returned to near pre-operative levels following steroid therapy which agrees with the findings of others (20, 21) and supports a mechanistic link with steroids.

The initial increase of HGF after transplantation might be due to the repair, growth, and remodeling that may result from transplantation. The lack of correlation with serum markers of inflammation as reported earlier (21) suggests that the change in HGF is not related directly to inflammatory or acute phase reactions; and the present finding that HGF serum levels peak moderately after but not prior to infection, is in concert with this invention.

Defrances and co-workers found that HGF was localised in bronchial epithelium of developing rats using immunohistochemical staining (22). They showed that HGF was produced by mesenchymal cells in each organ, which might regulate the epithelial cell growth and organ morphogenesis in a paracrine or endocrine manner (23). In the lung, the major sources of HGF are mesenchymal cells (macrophages, fibroblasts, and endothelial cells) (24). The elevated HGF levels in the transplanted patients could be associated with a reperfusion injury and endothelial cell production of HGF. Alternatively fibroblasts become activated during the early repair stages of bronchial and vascular anastomoses (25). HGF stimulates the proliferation of lung epithelial cells (22) and HGF may play an important role in bronchial epithelium replication (21). Together, this study shows that graft rejection but not infection results in early significant increased HGF levels and these levels decline after steroid treatment. As the present results show, HGF is a sensitive predictor of lung graft rejection.

### Comparative Example

### HGF in Renal Transplantation Rejection

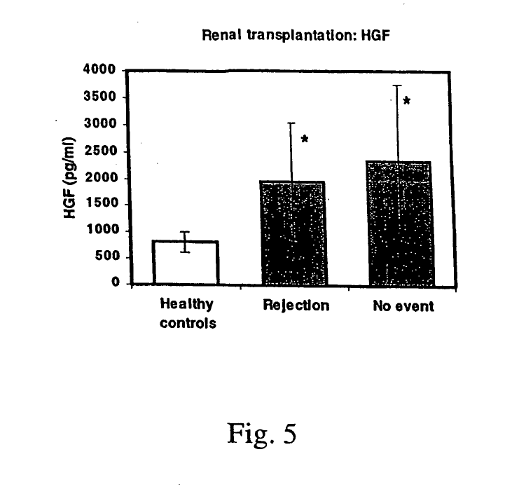

Patients who have undergone kidney transplantation were analysed with respect to their HGF level. 19 Patients were compared to 12 healthy controls. 12 patients had no events; 7 patients developed rejection. Mean HGF serum levels in controls, in patients after surgery with no events, and in patients with rejection were determined and are depicted in FIG. 5. The present comparative clinical data clearly show that HGF does not change in rejection vs. patients with no events after kidney transplantation (* p<0.05 vs. controls).

The comparison of the HGF serum levels in lung vs. kidney transplanted patients in the present invention at the one side, and the results shown for rat kidneys at the other, indicate the following. First, HGF is a pulmotrophic protein, and cells producing this protein further underline this fact, i.e. bronchial epithelial cells, fibroblasts and macrophages. The latter two cell types, especially macrophages, are involved in local and distant immune reactions, and are frequent residents of the lung tissue. Therefore, they can be easily stimulated to produce HGF due to lung graft rejection. Second, HGF can not be considered as a general marker for solid graft rejection, as indicated by the HGF levels in kidney transplantation rejection in this study. Beside the discussed role of the cells involved in HGF production and their significantly higher density in the lung vs. kidney, the well-known “hepato-pulmonary” pathway that acts both under physiologic and pathologic conditions (26) , might further explain the present findings. Finally, animal experiments, especially those in rodents, might not be a perfect measure of complex pathophysiologic processes that become activated in human transplantation rejections.

### REFERENCES

1. Toronto Lung Transplant Group. Unilateral lung transplantation for pulmonary fibrosis. N Engl J Med 1986;314:1140-1145.
2. Grossman et al., N Engl J Med 1990;322:727-733.
3. Pattersen et al., Current Topics in General Thoracic Surgery, Vol. 3, Lung Transplantation. Elsevier, Amsterdam, 1995, p. 363-378.
4. Arcasoy et al., N Engl J Med 1999;340:1081-1091.
5. Hosenpud et al., J Heart Lung Transplant 1999;18:611-626.
6. Bando et al., J Thorax Cardiovasc Surg 1995;109:49-59.
7. Williams T J. Rejection in lung transplantation. In: (Patterson G A and Couraud L, eds), Current Topics in General Thoracic Surgery, Vol. 3, Lung Transplantation. Elsevier, Amsterdam, 1995, p. 501-513.
8. Bewig et al., Clin Transplant 1999;13:432-439.
9. Khurshid et al., Am J Respir Crit Care Med 1998;157:A811.
10. Bewig et al., Eosinophilic alveolitis in BAL after lung transplantation. Transpl Int 1999;12:266-272.
11. Nakamura et al., Biochem Biophys Res Commun 1984;122:1450-1459.
12. Gohda et al., J Clin Invest 1988;81:414-419.
13. Rubin et al. A broad-spectrum human lung fibroblast-derived mitogen is a variant of hepatocyte growth factor. Proc Natl Acad Sci USA 1991;88:415-419.
14. Yanagita et al., J Biol Chem 1993;268:21212-21217.
15. Yoshimura et al., Transplant Proc 2000;32:2073-2074.
16. Yoshimura et al., Transpl Int 2002;15:156-162.
17. Mal et al., Chest 1998;113:645-651.
18. El-Gamel et al., J Heart Lung Transplant 1999;18:828-837.
19. Ross et al., J Heart Lung Transplant 1999;18:627-636.
20. Tsubouchi et al., Hepatology 1991;13:1-5.
21. Maeda et al., Am J Respir Crit Care Med 1995;152:1587-1591.
22. Defrances et al., Development 1992;116:387-395.
23. Matsumoto et al., Proc Natl Acad Sci USA 1992;89:3800-3804.
24. Yanagita et al., Biochem Biophys Res Commun 1992;182:802-809.
25. Hall et al., Am J Respir Cell Mol Biol 1992;7:49-57.
26. Schraufnagel et al., Clin Chest Med 1996; 17:1-15.
27. Zaccolo et al., Eur. J. Immunolo 1997; 27.
28. Gherardi et al., PNAS 2003; 100(21): 12039-12044.
29. Guirouilh et al., J. Of Hepatology 2001; 34: 78-83.
30. Ohnishi et al., Journal of Immunological Methods 2000; 244: 163-173.
31. Hata et al., The Journal of Histochemistry & Cytochemistry 2002; 50(11): 1461-1468.
32. Lassus et al., Pediatrics 2002; 110(4): 768-771.
33. Yango et al., Transplantation Proceedings 2002; 34: 3128-3129.
34. Kawasaki et al., Analyt. Biochem. 2003; 316: 15-22.
35. Jayasankar et al., Circulation 108 [suppl. II] 2003, II-230II-236.
36. Sengupta et al., Circulation 2003; 107: 2955-2961.
37. Khan et al., Fertility and Sterility 2003; 79: 173-181.
38. Heeschen et al., Circulation 2003; 107: 524-530.

1. Method for diagnosing lung transplantation rejection comprising determining the amount of Hepatocyte Growth Factor (HGF) in a body fluid or tissue sample of a patient who has undergone lung transplantation.

2. Method according to claim 1, characterized in that said body fluid or tissue sample is selected from the group comprising serum, plasma, blood, transsudates, lung or bronchial drainages or lavages, sputum, urine lung adjacent tissue and lung tissue.

3. Method according to claim 1, characterized in the following steps:
providing a body fluid or tissue sample of a patient who has undergone lung transplantation,
determining the amount of HGF in said body fluid or tissue sample,
comparing whether said determined amount of HGF is elevated in comparison with the amount of HGF in patients without lung transplantation rejection and
a) diagnosing lung transplantation rejection, if said determined amount of HGF is elevated in comparison with the amount of HGF in patients without lung transplantation rejection or
b) not-diagnosing lung transplantation rejection, if said determined amount of HGF is not elevated in comparison with the amount of HGF in patients without lung transplantation rejection.

4. Method according to claims 1, characterized in that said amount of HGF is determined by the use of a method or means selected from the group ELISA, RIA, mass spectroscopy, affinity chromatography, flow cytometry, protein-microarrays, fluorescent antibodies, HGF binding substances, especially monoclonal and polyclonal HGF-antibodies or HGF receptors, immunohistochemical methods, immunoprecipitation, Western blot, fluorescence resonance energy transfer (FRET), cDNA array and nucleic acid amplification methods.

5. Method according to claims 1, characterized in that an elevated HGF amount being indicative of lung transplantation rejection is ascertained by at least one of the following:
the determined HGF amount is at least 400%, preferably at least 500%, especially at least 600%, of the mean baseline level of HGF in healthy controls,
the determined HGF amount is at least 200%, preferably at least 300%, especially at least 350%, of the mean baseline level of HGF in patients before lung transplantation,
the determined HGF amount is at least 200%, preferably at least 300%, especially at least 350%, of the mean baseline level of HGF in patients on day 3 or later after lung transplantation with no events, especially with no transplantation rejection and/or with no infection.

6. Method according to claim 5, characterized in that
the mean baseline level of HGF in healthy controls is between 300 and 900 pg/ml serum or
the mean baseline level of HGF in patients before lung transplantation is between 900 and 1600 pg/ml serum or
the mean baseline level of HGF in patients on day 3 after lung transplantation with no events, especially with no transplantation rejection events and/or with no infection is between 800 and 1500 pg/ml serum.

7. Method according to claims 1, characterized in that an elevated HGF amount being indicative of lung transplantation rejection is ascertained by a serum level at day 3 or later after lung transplantation of higher than 2000 pg/ml, preferably higher than 2300 pg/ml, especially higher than 2500 pg/ml.

8. Use of HGF binding substances for measuring the amount of HGF in a body fluid or tissue sample of a patient who has undergone lung transplantation provided from a patient who has undergone lung transplantation.

9. Kit for diagnosing lung transplantation rejection comprising
a sample container with a body fluid or tissue sample of a patient who has undergone lung transplantation and
means for determining the amount of HGF in said sample.

10. Kit according to claim 9, characterized in that it further comprises comparative HGF preparations containing a determined amount of HGF.

11. Kit according to claim 9, characterized in that said means for determining the amount of HGF comprises a (micro) plate, biochip or membrane.
